(19) United States
(12) Patent Application Publication
(10) Pub. No.: US 2026/0014341 A1
(43) Pub. Date: Jan. 15, 2026

(54) 1. Scent Diffusion Device for Supplemental Oxygen

(71) Applicants: Paul Carey, GRAND HAVEN, MI (US); Drew Carey, GRAND HAVEN, MI (US)

(72) Inventors: Paul Carey, GRAND HAVEN, MI (US); Drew Carey, GRAND HAVEN, MI (US)

(21) Appl. No.: 18/770,034

(22) Filed: Jul. 11, 2024

(51) Int. Cl. A61M 16/10; A61M 16/00; A61M 16/06; A61M 21/00

(52) U.S. Cl. CPC A61M 16/10; A61M 16/0003; A61M 16/0672; A61M 2021/0016; A61M 2202/0007; A61M 2202/0208; A61M 2205/121; A61M 2205/3331

(57) ABSTRACT

Embodiments of a scent diffusing device for enhancing the olfactory experience of users receiving supplemental oxygen are disclosed herein. The device comprises a scent diffuser housing, a scent diffusion mechanism within the housing, and connectors for integration with standard oxygen tubing systems. The scent diffuser housing accommodates a scent diffusion mechanism, which may include a diffuser pad saturated with scent oil. The housing is designed to integrate with standard oxygen tubing systems used in medical facilities and home healthcare settings. The device can be configured to connect directly to the oxygen source via standard oxygen tubing or to be placed in the airflow between the oxygen source and the user's nasal cannula or mask. Importantly, no modification of the existing oxygen tubing, machine, or mask is required for installation. The scent diffusing device offers a solution for delivering scent to individuals using supplemental oxygen, thereby enhancing their sensory experience.

### CROSS-REFERENCE TO RELATED APPLICATIONS

We, Paul Carey and Drew Carey, the inventors and applicants herein, hereby claim the benefit of priority to the provisional patent application identified as 63/526,851, filed on Jul. 14, 2023, entitled “Scent diffusing device to deliver scent into Oxygen tubing”. The entire disclosure of said provisional patent application, including all descriptions, drawings, and any other supporting materials, is hereby expressly incorporated by reference into the present utility patent application. This utility patent application is filed within 12 months of the filing date of the aforementioned provisional patent application, in accordance with 35 U.S.C. § 119 (e). The present utility patent application covers the same invention disclosed in the provisional patent application and claims priority thereto for all subject matter disclosed therein.

### TECHNICAL FIELD

The present invention generally relates to the field of scent diffusion, specifically relating to the diffusion of scent into supplemental oxygen systems.

### BACKGROUND OF THE INVENTION

Supplemental oxygen is essential for individuals in medical facilities or at home, yet traditional scent diffusion methods are ineffective due to the direct airflow of oxygen delivery systems. Current solutions lack the capability to deliver scents directly to users of supplemental oxygen and restrict olfactory experiences for individuals requiring supplemental oxygen. The use of open flames or heating elements in existing scent diffusers poses significant safety risks in environments where supplemental oxygen is present. The potential for ignition or overheating near oxygen sources creates a hazardous situation for users and those around them.

This invention addresses these challenges by facilitating direct and efficient scent diffusion for users of supplemental oxygen, enhancing their sensory experience in hospital, care facility, or home environments where traditional methods are impractical or unsafe.

### SUMMARY OF THE INVENTION

In the present disclosure, all embodiments serve as illustrative and non-limiting representatives of numerous possible embodiments. Described herein are embodiments providing a device for in line scent diffusion to a supplemental oxygen system. This device may comprise a hollow scent diffuser housing, a scent diffuser pad saturated with scent oil, and standard oxygen tubing connectors. This device may be configured to connect in line between the oxygen supply and nasal cannula, with no modification of existing oxygen supply hoses or nasal cannulas necessary. Oxygen flows from the supply into the scent diffuser housing, past the diffuser pad saturated with scent oil, diffusing scent into the oxygen, and out to the user through the nasal cannula.

In some embodiments, the hollow scent diffuser housing may comprise of two parts connected via male and female threading, enclosing the diffuser pad saturated with scent oil, with each part featuring a connector for oxygen tubing or a nasal cannula.

In some embodiments, the scent oil may saturate the diffuser pad, typically made of cotton or another absorbent material. The hollow scent diffuser housing features oxygen tubing and nasal cannula connectors on each side while the diffuser pad is enclosed within.

In some embodiments, the hollow scent diffuser housing may feature holders or grooves to secure the diffuser pad in position.

To diffuse scent into the oxygen supply of a supplemental oxygen system, oxygen flows from a tank or wall mount, through a supply hose, and a nasal cannula or mask. A scent diffuser device is inserted in line between the oxygen supply and nasal cannula, with the diffuser pad saturated with scent oil beforehand. This scent oil, which may be essential oils or other aromatic substances, enhances the overall sensory experience of users by diffusing a pleasant scent in line while oxygen flows through the device.

The primary objective of this invention is to deliver scent in line to individuals using supplemental oxygen, thereby enriching their sensory experience. The distinctive features believed to be novel are particularly outlined and claimed in the concluding portion of the specification. Further details, aspects, and advantages of this invention will be elucidated with reference to the following drawings and detailed description.

### BRIEF DESCRIPTION OF THE DRAWINGS

For a comprehensive understanding of the present invention, refer to the following detailed description and accompanying figures in which:

FIG. 1 is a perspective view of an embodiment showing an assembled scent diffusion device;

FIG. 2 is a side view of the assembled scent diffusion device of FIG. 1;

FIG. 3 is an exploded view of the scent diffusion device of FIG. 1 and the application of scent oil;

FIG. 4 shows a cross-sectional exploded view of the scent diffusion device of FIG. 3;

FIG. 5 shows an exemplary embodiment of the scent diffusion device of FIG. 1 configured in line with a supplemental oxygen system;

FIG. 6 shows a cross-sectional side view of an exemplary embodiment of the process of scent diffusion through the assembled scent diffusion device of FIG. 1.

### DETAILED DESCRIPTION OF THE INVENTION

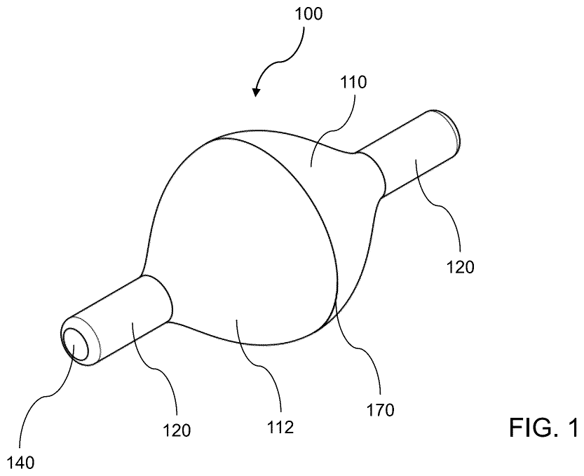

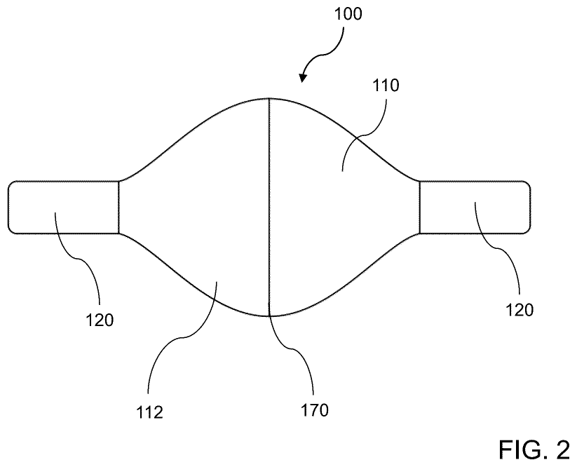

The following description relates to a scent diffusion device designed for integration with supplemental oxygen systems. In the embodiment shown in FIG. 1 and FIG. 2, the scent diffuser housing 100 consists of two parts: a female housing 110 and a male housing 112, which connect via male thread 124 and female thread 122 to create an airtight seal 170. This design ensures secure enclosure of the diffuser pad 134 while maintaining easy access for replacement or application of scent oil 150 to the diffuser pad 134. Both the female housing 110 and male housing 112 have a standard oxygen tube connector 120 on the non-threaded end for connecting in line with a supplemental oxygen system when assembled. Each standard oxygen tube connector 120 consists of a hollow opening 140 to allow oxygen flow o through the scent diffuser housing 100.

The diffuser pad 134 is placed within the scent diffuser housing 100 and held in position by diffuser pad holders 130. The diffuser pad holders 130 are designed with legs 132 that keep the diffuser pad 134 securely positioned within the scent diffuser housing cavity 142 without obstructing the oxygen flow o. The diffuser pad 134 is typically made of an absorbent material such as cotton, which can effectively retain and gradually diffuse the scent s of the scent oil 150 into the oxygen flow o.

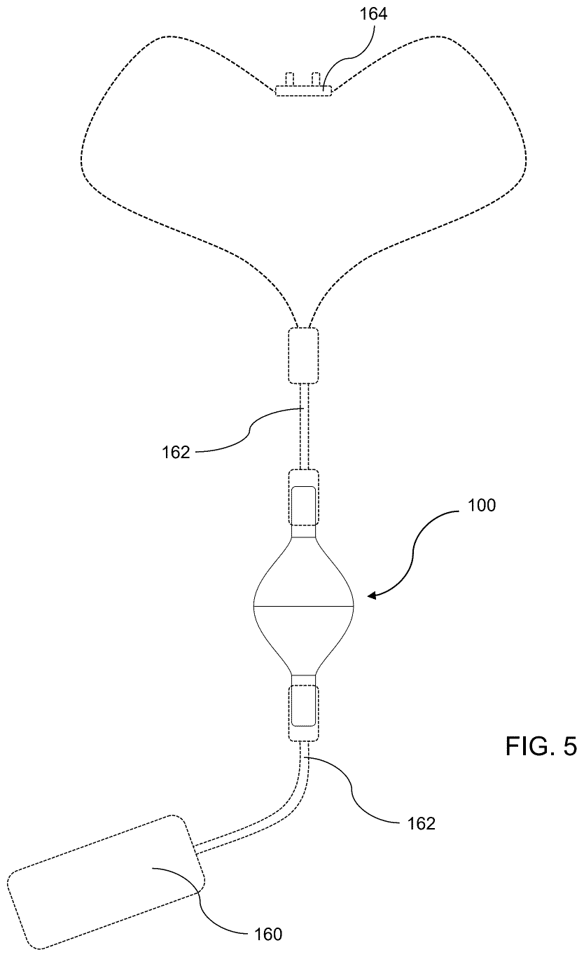

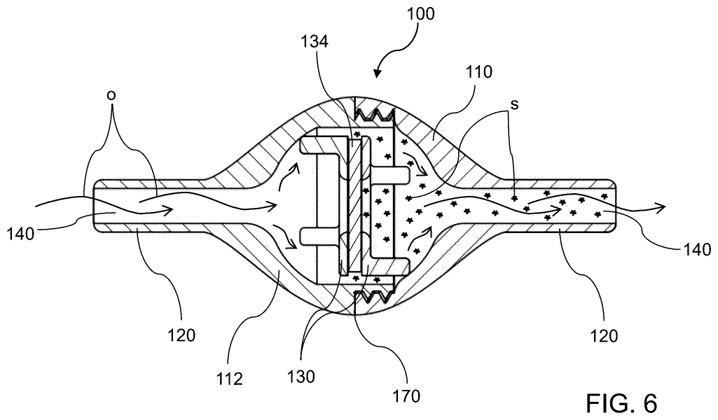

The exemplary embodiment of FIG. 5 shows how the device is configured to be connected in line between an oxygen supply 160 and a nasal cannula 164 without requiring any modifications to the existing standard oxygen tubing 162 or nasal cannula 164. The oxygen flow o from the oxygen supply 160 through standard oxygen tubing 162 into the scent diffuser housing 100 via standard oxygen tube connector 120, past the diffuser pad 134, saturated with scent oil 150 and positioned within the scent diffuser housing cavity 142, and exits the scent diffuser housing 100 with scent s to the nasal cannula 164 of the user. As shown in FIG. 6, this process allows the scent s to be diffused into the oxygen flow o, enhancing the user's sensory experience while using supplemental oxygen.

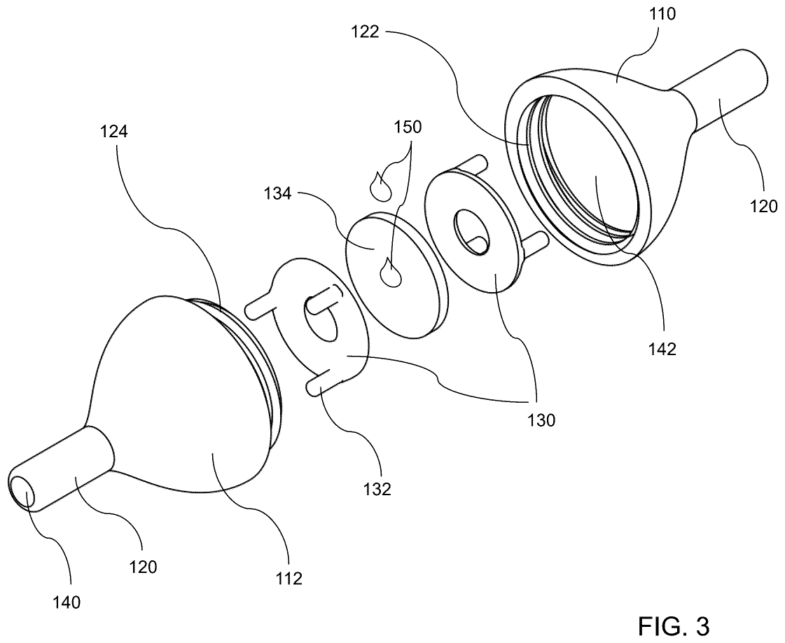

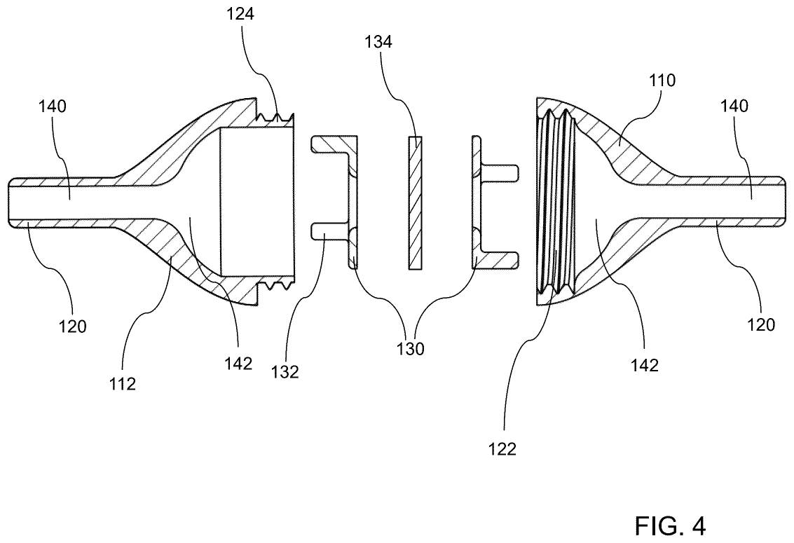

The embodiment of FIG. 3 and FIG. 4 show the components of the scent diffuser housing 100 and the orientation for assembly. The female housing 110 and male housing 112 contain two diffuser pad holders 130 and a diffuser pad 134 positioned between them in the scent diffuser housing cavity 142. The diffuser pad 134 is saturated with scent oil 150, which may be essential oils or other aromatic substances.

The following description relates to the assembly of the scent diffuser housing 100. First, prepare the diffuser pad 134 by saturating it with the chosen scent oil 150, ensuring that the pad is evenly saturated but not excessively wet to avoid dripping. The user can adjust the amount of scent oil 150 applied to the diffuser pad 134 to achieve the desired concentration of scent s in the oxygen flow o. Next, insert the saturated diffuser pad 134 into the scent diffuser housing 100 by placing it in the female housing 110, ensuring that it is positioned correctly and held securely by the diffuser pad holders 130. The legs 132 of the diffuser pad holders 130 keep the diffuser pad 134 in place without obstructing oxygen flow o. Once the diffuser pad 134 is in position, connect the female housing 110 and male housing 112 by aligning the male housing 112 with the female housing 110 and screwing them together using the male thread 124 and female thread 122, creating the airtight seal 170. Next, connect the scent diffuser housing 100 to the oxygen supply 160 and the nasal cannula 164 using the standard oxygen tube connector 120 on each side of the housing. Finally, ensure that all connections are secure, that there are no leaks, and that the scent diffuser housing 100 is firmly attached to both the oxygen supply 160 and the nasal cannula 164. Once assembled, the device is ready for use.

What is claimed is:

1. A device for diffusing scent into a supplemental oxygen system, comprising: a hollow housing with an oxygen tubing connector on each side; a diffuser pad positioned within the hollow housing; wherein the diffuser pad is saturated with scent oil; wherein the device is configured to be connected in line between an oxygen supply and a nasal cannula without requiring modification to the existing oxygen tubing or nasal cannula; wherein oxygen flows from the oxygen supply into the housing, past the diffuser pad saturated with scent oil, and exits to the user through the nasal cannula.

2. The device of claim 1, wherein the hollow housing comprises two halves connected via male and female threading.

3. The device of claim 1, wherein the diffuser pad is formed of an absorbent material.

4. The device of claim 1, wherein the scent oil used to saturate the diffuser pad are essential oils or other aromatic substances.

5. The device of claim 1, further comprising two diffuser pad holders within the hollow housing for holding the diffuser pad in position.

6. The device of claim 1, wherein a lid is removably attachable to the hollow housing to allow replacement or saturation of the diffuser pad.

7. The device of claim 1, wherein the device is configured to maintain a continuous flow of oxygen without significant pressure drop or disruption.

8. The device of claim 1, wherein a variety of scent oils can be applied to the diffuser pad.

9. The device of claim 1, wherein a varying amount of scent oil can be applied to the diffuser pad.

10. A method of diffusing scent into the oxygen supply of a supplemental oxygen system, comprising: providing oxygen flow from a tank or wall mount through a supply hose and nasal cannula; inserting a scent diffuser device in line between the oxygen supply and the nasal cannula; saturating the diffuser pad within the scent diffuser housing cavity with scent oil prior to use; allowing the oxygen to flow through the scent diffuser housing, past the saturated diffuser pad, and delivering it to the user through the nasal cannula.
